(19) United States
(12) Patent Application Publication
(10) Pub. No.: US 2018/0256380 A1
(43) Pub. Date: Sep. 13, 2018

(54) ORTHOPEDIC JOINT DEVICE

(71) Applicant: OTTO BOCK HEALTHCARE GMBH, Duderstadt (DE)

(72) Inventors: Martin PUSCH, Duderstadt (DE); Karl-Otto STORCK, Duderstadt (DE)

(73) Assignee: OTTO BOCK HEALTHCARE GMBH, Duderstadt (DE)

(21) Appl. No.: 15/762,683

(22) PCT Filed: Sep. 5, 2016
(86) PCT No.: PCT/EP2016/070865

§ 371 Date: Mar. 23, 2018

(30) Foreign Application Priority Data
Sep. 24, 2015 (DE) 10 2015 116 149.4

(51) Int. Cl. A61F 5/01

(52) U.S. Cl. CPC A61F 5/0123; A61F 5/013; A61F 2/64; A61F 2005/0169; A61F 2005/0155; A61F 2005/0158

(57) ABSTRACT

An orthopedic joint device having an upper part, on which upper connecting members for fastening to a patient are arranged, and a lower part, which is fastened to the upper part with articulation about a pivot axis. A flexion-moment-controlled halting device is arranged between the upper part and the lower part, which halting device blocks flexion movement and, when a predetermined flexion moment is exceeded, releases the flexion movement.

The invention relates to an orthopedic joint device having an upper part, on which are arranged upper connection means for securing to a patient, and having a lower part, which is fastened on the upper part in an articulated manner about a pivot pin. The orthopedic joint device is designed, in particular, in the form of an artificial prosthetic knee joint or orthotic knee joint or prosthetic or orthotic elbow joint, but is not restricted to these embodiments. An embodiment in the form of a locking knee joint provides for standing and, possibly, walking with a locked joint; once the latter has been released, however, the upper part can be pivoted relative to the lower part. However, it is also possible for the joint device to be used in the form of a so-called dynamic joint, which counteracts spastic contractions or can be used to treat the contractions by means of stretching exercises.

So-called locking knee joints are often used in relatively immobile patients in whom mobility may have been restricted, for example, as a result of diseases of the lower extremities. Restricted mobility here means that there is a need merely for knee joints which provide for restriction-free sitting and ensures the greatest possible level of reliability when the patient is at all mobile. For this purpose, these knee joints provide usually just two states, that is to say a locked, extended position and an unlocked position, in which the knee joint can be moved freely. No provision is made for adaptation of the damping in a swing phase or stance phase. Such a knee joint in the form of a prosthetic knee joint is described in DE 20 2006 007 641 U1. In order to unlock such a prosthetic knee joint, it is necessary to actuate an unlocking mechanism by hand.

DE 10 2008 024 747 A1 relates to an orthopedic device having a joint which connects an upper part to a lower part in a pivotable manner. Connection means for fastening a limb are arranged on the upper part. A locking device prevents a flexing movement of the upper part relative to the lower part, wherein the locking device can be actuated in active fashion by the person using the orthopedic device. A control device is assigned to the locking device, and this is connected to at least one sensor on the orthopedic device, and therefore the locking device is automatically unlocked or arrested in dependence on the sensor signal.

DE 103 51 916 A1 relates to a prosthetic knee joint having an upper part and a lower part, which is mounted in a pivotable manner on the upper part, and a resistance device, which is arranged between the upper part and lower part, provides locking against a flexion movement and, in dependence on a knee angle, arrests flexion in a defined angle range and provides for free movement capability outside the angle range.

DE 20 2004 008 014 U1 relates to a swing-phase-control means for an artificial knee joint, having a piston/cylinder unit with two chambers and two throttles for throttling or arresting through-passage of a fluid from the first chamber into the second chamber. Devices are present in order to close the throttles when a certain pressure is reached in the chambers.

It is the object of the present invention to provide an orthopedic joint device, e.g. an artificial knee joint, which can be unlocked with an increased level of reliability and provides for adaptation of the functionality to the essential use states.

This object is achieved according to the invention by an orthopedic joint device having the features of the main claim. Advantageous embodiments and developments of the invention are disclosed in the dependent claims, the description and the figures.

The orthopedic joint device having an upper part, on which are arranged upper connection means for securing to a patient, and having a lower part, which is fastened on the upper part in an articulated manner about a pivot pin, provides for a flexion-moment-controlled retaining device to be arranged between the upper part and the lower part, said retaining device arresting flexion and, when a predetermined flexion moment is exceeded, releasing the flexion. Conventional locking joints provide form-fitting locking of the upper part in relation to the lower part, and therefore the locking between the upper part and the lower part is maintained irrespective of a flexion moment applied. It is only by virtue of a form-fitting locking device, for example a slide, a hook or a pin, being unlocked that the orthopedic joint device, which may be designed, in particular, in the form of a prosthetic knee joint or orthotic knee joint or prosthetic or orthotic elbow joint, is unlocked and accessible for flexion. Provision is made, in principle, for free flexion to be made possible following unlocking, that is to say once the retaining force has been overcome, by the application of a predefined flexion moment about the axis of rotation of the orthopedic joint device, and there is therefore no obstruction or restriction to the flexion movement. Free flexion is not intended to mean completely resistance-free flexion, since this is not possible in technical systems. Free flexion is present even when there are flexion resistances present on account of frictional resistances. In the case of the embodiment in the form of an artificial knee joint, the user can use both hands for safeguarding purposes as he sits down, and this allows the user to feel more confident. If the orthopedic joint device is not used in the form of an artificial knee joint, any orthosis or prosthesis can be equipped with a corresponding retaining device in order to arrest flexion up to a predetermined moment and, so as to safeguard against overload, to release the joint once a flexion moment has been exceeded.

As long as the retaining device is active, for example by way of a form-fitting, force-fitting or some other mechanical locking mechanism, the joint or the orthopedic joint device remains locked, that is to say that in the case of conventional loading, e.g. when the user is standing or walking or the elbow is in a holding position, flexion, that is to say pivoting of the upper part relative to the lower part about the pivot pin, is not possible. It is only by application of force to the heel or the lower part in general, or by application of a flexion moment which acts about the pivot pin, that the retaining device is deactivated, and therefore the locking mechanism in the artificial orthopedic joint device is disengaged and flexion is possible.

A development of the invention provides for a damper device to be arranged between the lower part and the upper part and to damp a flexion movement and/or extension movement of the lower part relative to the upper part. In addition to the damping which is immanent in the system, a separate damping device is therefore provided, and this separate device, following the disengagement of the locking mechanism, provides damping of the pivoting movement, in particular damping in the flexion direction, in order thus to assist a user in sitting down. In the case of an embodiment in the form of a dynamic joint, the movement is damped once a flexion moment has been exceeded, and this therefore prevents the situation where, once the orthopedic joint device has been released, unbraked flexion movement, for example in the event of a spasm, to maximum flexion takes place.

The damper device can be designed such that it can be switched between a high damping resistance and a lower damping resistance, so as to be able to adapt to the needs in any given situation. For example, it may be desirable to provide a high level of damping in order to assist a sitting-down movement or to brake some other movement, whereas, in the seated or flexed state, a comparatively low level of damping can be established in order for it to be possible for the user to perform comparatively small movements in the leg or arm without hindrance while he is seated.

The damper device may be assigned an axial-force-dependent switching device which, when the axial force acting on the lower part drops below a certain value, enables a reduced damping resistance. When the user sits down, a comparatively high axial force acts on the lower leg along the longitudinal extent of the lower leg, for example of a lower-leg splint or a lower-leg tube. A similar loading situation arises when the user uses his hand for support as he sits down; in this case, the axial force acts in the forearm splint or in the forearm tube. This comparatively high axial force is an indicator of a movement situation in which a comparatively high damping resistance is desired. If this axial force falls away, for example while the user is seated, in the case of a lower leg being raised or of a forearm being relieved of loading, a reduced damping resistance is enabled and a free, or more or less free, flexion movement and extension movement is made possible.

The damper device may be assigned a mechanical switch or a sensor-controlled switch provided with an actuator, and therefore switching can be implemented either purely mechanically or electromechanically with a motor-driven actuator.

The retaining device may be designed in a force-fitting manner, for example in the form of a magnet or suction cup. As an alternative, it is possible to use form-fitting retaining devices, for example touch-and-close fasteners, leaf-spring elements, which slide along a roller, or a hydraulic unit. In the case of a hydraulic unit, it is possible, up to a predetermined flexion moment, for overflow from a flexion chamber into an extension chamber to be arrested, for example by a preloaded nonreturn valve, and it is only when an applied moment is exceeded that the overflow channel is released, this resulting in flexion, possibly in combination with an increased damping resistance, being released. It is advantageously possible to adjust the prestressing of the nonreturn valve, and therefore the release moment.

It is possible to adjust the entire amount of retaining force exerted by the retaining device, in order to avoid flexion of the orthopedic joint device, so that the artificial joint can be adapted to the respective patient.

A development of the invention provides for the upper part to be mounted in a displaceable manner in relation to the lower part and for deactivation of the retaining device to be possibly only at a certain position of the upper part in relation to the lower part in the axial direction. For this purpose, provision may be made for the upper part to be spring-loaded in relation to the lower part, that is to say for a displacement-counteracting spring element to be arranged between the upper part and the lower part, said spring element allowing pivoting only when a certain position is assumed.

The retaining device may have at least one magnetic coupling and/or a suction-cup coupling and/or a snap spring and/or a hydraulic arresting unit with a switchable arresting valve.

In addition to the retaining device, a separate hydraulic damper may be arranged between the upper part and the lower part in order to damp an extension movement and/or flexion movement of the upper part relative to the lower part to a desired extent once the retaining moment has been overcome.

The retaining device may have, or form, a sensorless-operation release mechanism which is part of the retaining device or is formed by the retaining device. This makes it possible, on the one hand without electrical or electronic components, to provide a retaining function, which prevents a joint from flexing in an undesired manner, and, on the other hand without any outlay in terms of control or regulation and/or driven actuators or sensors, to provide for release of the locking mechanism as from a predetermined, adjustable flexion moment. The release mechanism can be integrated in the retaining device and be realized by a limited retaining force. It is thus possible for a magnetic holder or a suction cup to provide, at the same time, a retaining function and a release function by way of the limited retaining force. A spring-loaded lever or a spring can retain a catch in the locking position until, in the case of a predetermined flexion moment, the spring loading is no longer sufficient to retain the catch, or some other form-fitting or force-fitting arresting device in the locking position. Without any further drive power or any further signal, the retaining function is then terminated and the joint is released. Both the retaining device and the release mechanism operate mechanically and can function without sensors or a control device.

Exemplary embodiments of the invention will be explained hereinbelow with reference to the accompanying figures, in which:

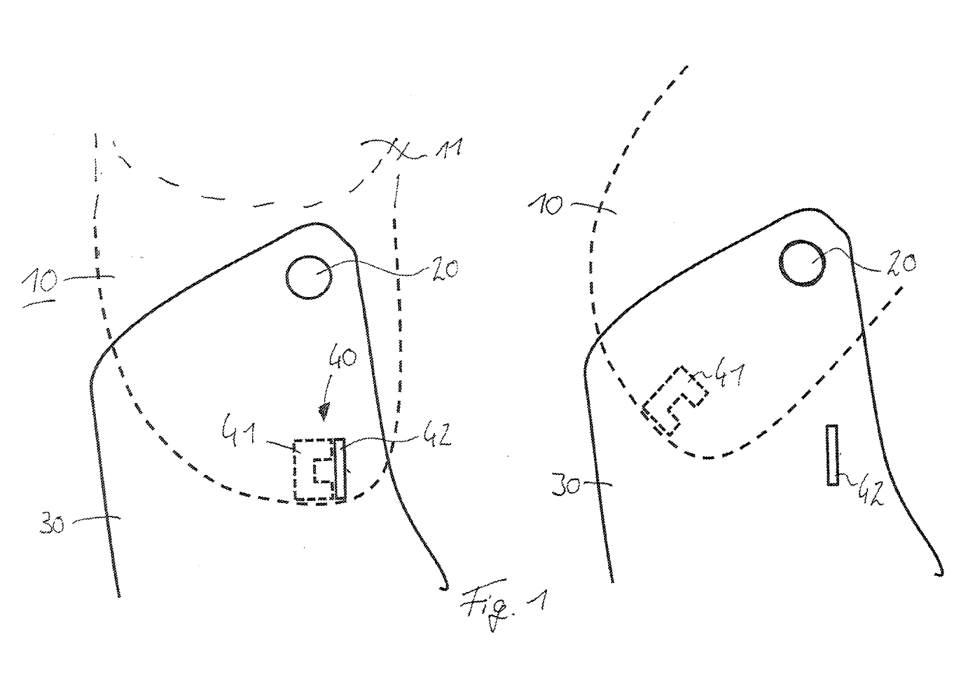

FIG. 1 shows a schematic illustration of an orthopedic joint device having a magnetic retaining device;

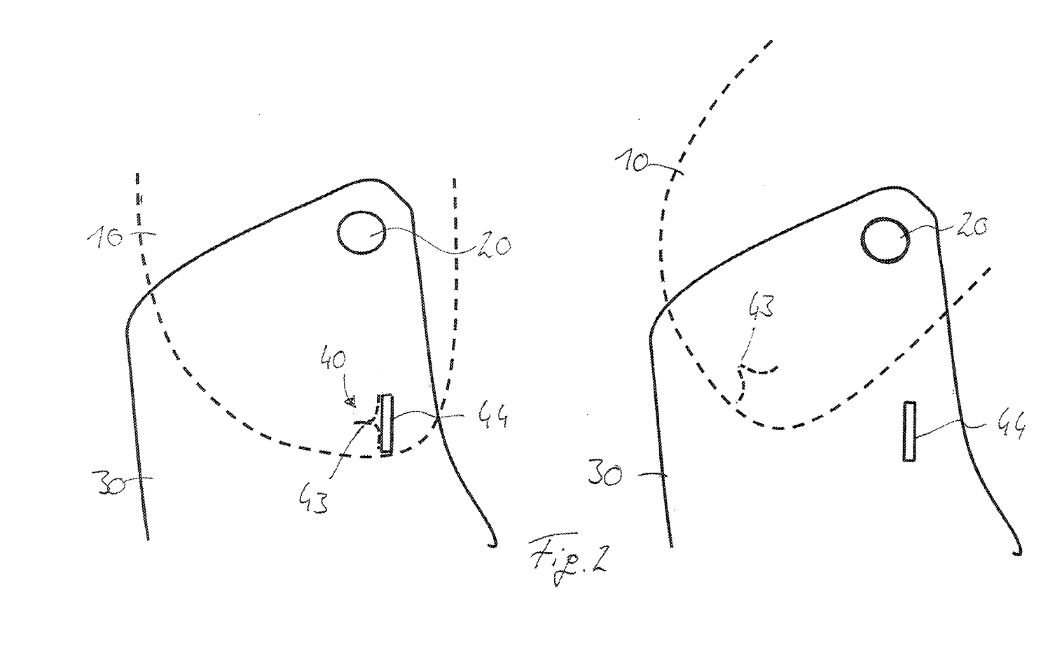

FIG. 2 shows a variant of FIG. 1 having a negative-pressure-based retaining device;

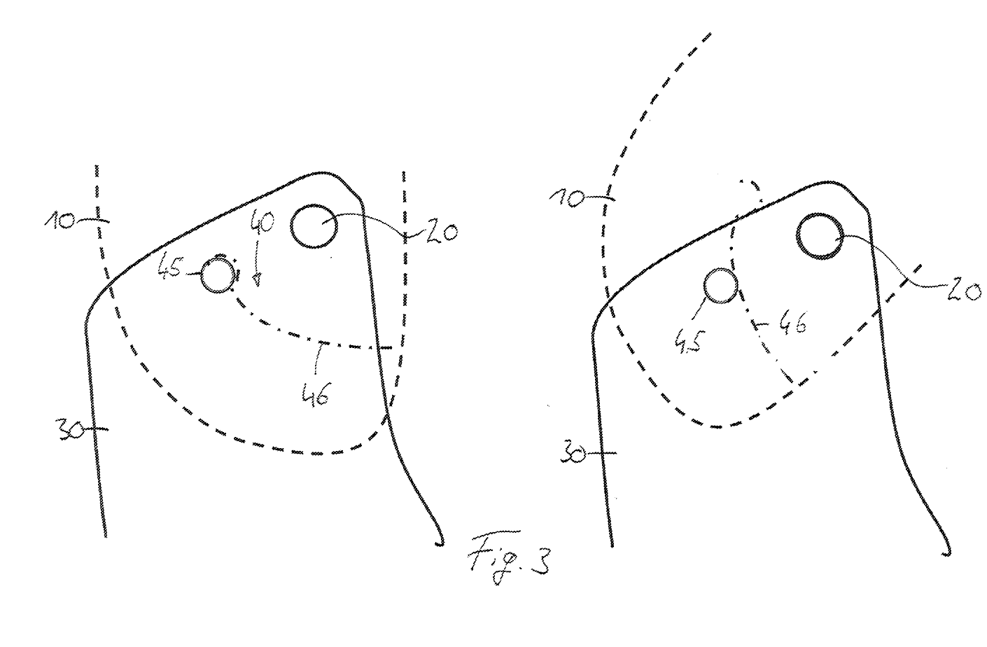

FIG. 3 shows a variant of the invention having a spring-loaded retaining device;

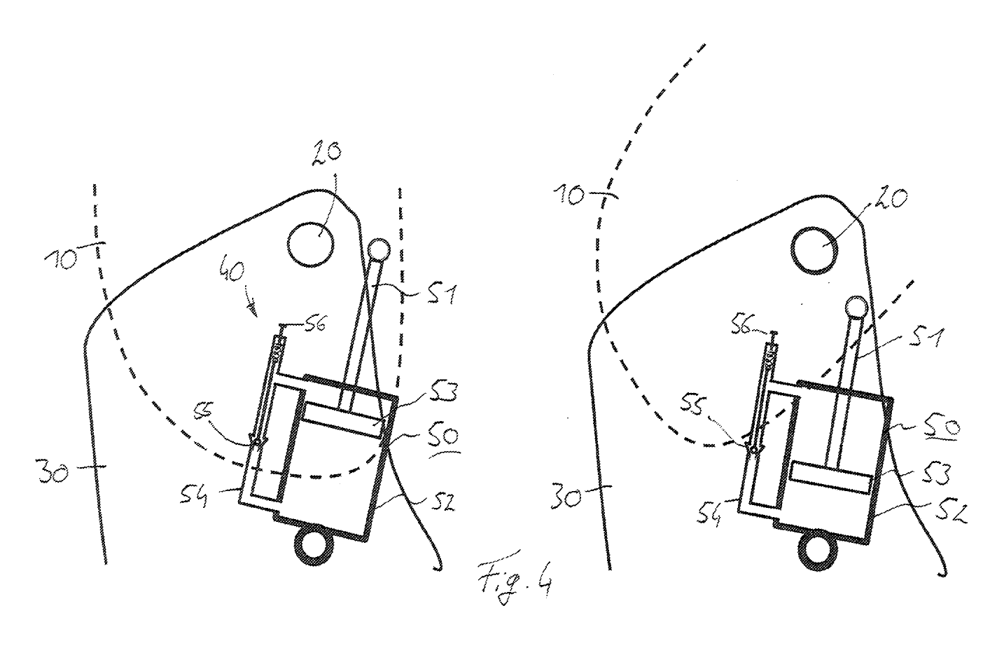

FIG. 4 shows a variant of the invention having a hydraulic retaining device;

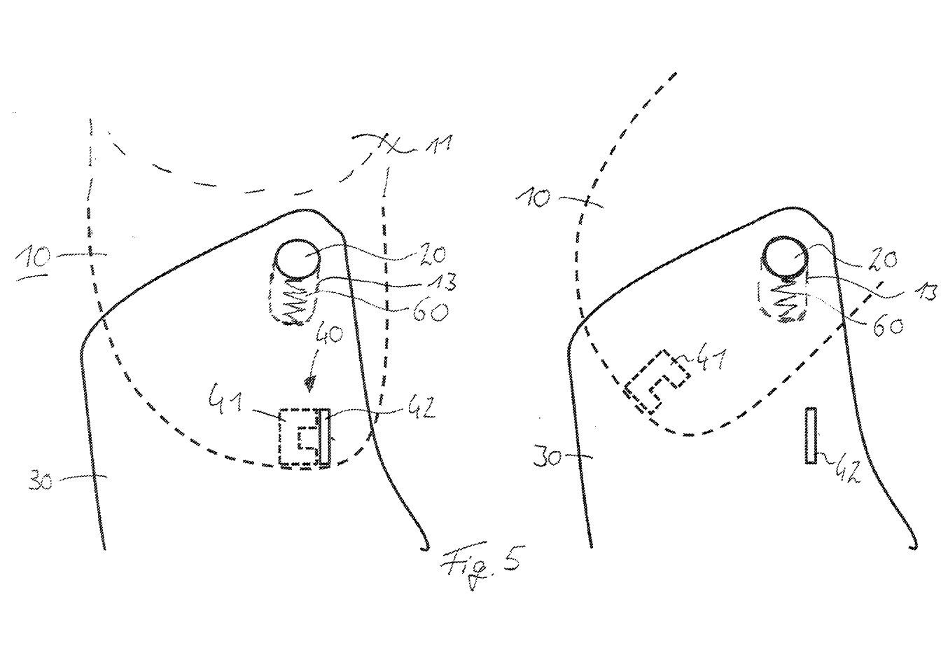

FIG. 5 shows a further variant of FIG. 1; and

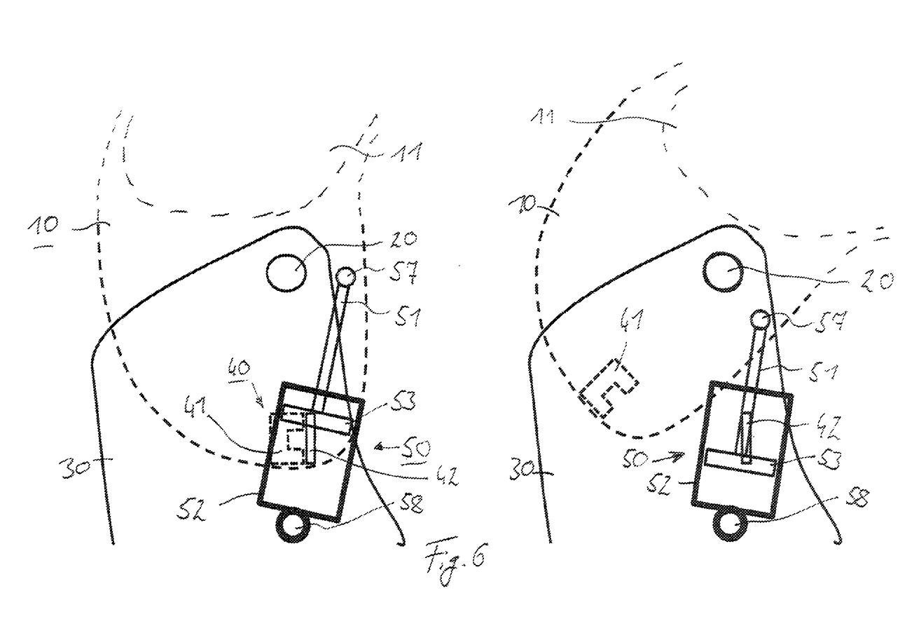

FIGS. 6-8 show variants of FIGS. 1 to 3 having an additional hydraulic damper.

FIG. 1 shows a schematic sectional illustration of an orthopedic joint device in the form of an artificial knee joint having an upper part 10, on which are arranged upper connection means 11 in the form of an upper-leg socket for securing to a patient. As an alternative to an embodiment in the form of a prosthetic knee joint, the artificial knee joint may also be designed in the form of an orthotic knee joint in which the upper part 10 is designed in the form of an upper-leg splint and the upper connection means are designed in the form of a cuff, tab, strap or the like, in order for the orthosis to be fitted to the patient's upper leg by way of the upper-leg splint. As an alternative to an embodiment in the form of an artificial knee joint, other uses are provided with the orthopedic joint device and are covered by the invention, for example for prostheses and/or orthoses for upper and lower extremities such as ankle orthoses, elbow orthoses, wrist orthoses and prostheses for ankles, knees, elbows or hands.

A pivot pin 20 is formed on the upper part 10, and a lower part 30, which is secured on the upper part 10, is mounted such that it can be pivoted about said pivot pin 20. The lower part 30 may be designed in the form of a lower-leg splint with fastening means for securing the lower-leg splint to the lower leg, in the case of an embodiment in the form of an orthosis. The fastening means may be designed in the form of straps, cuffs or the like and wrap around the lower leg; if appropriate, a foot part may be arranged on the lower-leg splint, to form a KAFO (Knee Ankle Foot Orthosis). In the case of the artificial knee joint being embodied in the form of a prosthetic knee joint, the lower part 30 is designed in the form of a lower-leg tube, on which a prosthetic foot (not illustrated) is secured. Upper-extremity prostheses or ortheses are constructed in a corresponding manner.

The pivot pin 20 is designed in the form of a continuous pivot pin; it is also possible, in principle for a multi-link joint to form a non stationary joint pin.

The upper part 10 projects distally beyond the pivot pin 20 and has a magnet 41 fastened on it, the magnet butting against a yoke 42, which is fastened on the lower part 30. The magnet 41 and the yoke 42 together form the retaining device 40, via which the knee joint is locked in the extended position illustrated. In such a locked position, it is not possible for the lower part 30 to pivot relative to the upper part 10 about the pivot pin 20; the user or patient can trust in the extended knee joint being secured when he is standing, walking or sitting.

The right-hand illustration of FIG. 1 shows to the knee joint in a flexed, bent position. Application of a flexion moment about the pivot pin 20, for example when a hip moment is applied or a heel is placed down with sufficient loading of a ground-reaction force vector running behind the pivot pin 10, causes the retaining force of the retaining device 40 to be exceeded, and therefore the magnet 41 disengages from the yoke 42 and the upper part 10 can be pivoted freely relative to the lower part 30 about the pivot pin 20.

The exemplary embodiment illustrated in FIG. 1 shows no further damping component or damping device; it is also possible, in principle, for the upper part 10 and the lower part 30 to have provided, and arranged, between them a damper device which damps a flexion movement and/or extension movement of the upper part 10 relative to the lower part 30. Such a damper device is advantageously designed in the form of a hydraulic damper or pneumatic damper; it is also possible, in principle, for a solid damper to be arranged between the upper part 10 and the lower part 30.

FIG. 2 illustrates a variant of FIG. 1, the component bearing the same reference signs corresponding to one another. In contrast to FIG. 1, the retaining device 40 is of vacuum-based construction in FIG. 2, the retaining force being realized via a vacuum effect. The upper part 10 has fastened on it a suction cup 43, which butts against a smooth surface 44, which is formed or arranged on the lower part 30, and provides for fixed abutment via a negative pressure in the enclosed volume. It is only when a predeterminable flexion moment is exceeded that the suction cup 43 is disengaged from the smooth surface 44, and ambient air enters into the volume between the suction cup 43 and the smooth surface 44 and results in pressure equalization, the ambient air therefore no longer keeping the suction cup 43 pressed against the smooth surface 44. The retaining force provided by the suction cup 43 is overcome and the knee joint is released for the flexing movement.

FIG. 3 shows a variant of the invention in which the retaining device 40 is of resilient design and comprises a roller 45 or a bolt, which is fastened on the lower part 30, and a leaf spring 46, which is arranged on the upper part 10. At a front end, the leaf spring 46 has a convexity or shaping which is designed to correspond to the contour of the roller 45, and therefore, in the locked state according to the left-hand illustration of FIG. 3, the knee joint is locked in a quasi form-fitting manner. On account of prestressing, the spring 46 pushes against the roller 45, and therefore the joint, e.g. a knee joint, elbow joint, ankle joint or wrist joint in the form of an orthosis or prosthesis, is retained in the extended position since the convexity or curvature of the spring 46 accommodates the roller 45. When a sufficiently high flexion moment is applied about the pivot pin 20, the curvature or convexity at the end of the leaf spring 46 moves out of the roller 45, the roller 45 rolls on the spring 46 and the joint can flex essentially freely.

The embodiments illustrated according to FIGS. 1 to 3 may also have the components of the retaining device 40 arranged the other way round. In this case, the magnet 41 is arranged on the lower part 30 and the yoke 42 is arranged on the upper part 10, the smooth surface 44 is located on the upper part 10 and the suction cup 43 is located on the lower part 30, just as the spring 46 is located on the lower part 30 and the roller 45 is located on the upper part 10. Provision is likewise made for the retaining force of the retaining device 40 to be designed to be adjustable in each case; in the case of a magnetic retaining device, this can be achieved by the distance between the yoke 42 and magnet 41 being increased, by material being introduced, or else by the contact surface area between the magnet 41 and the yoke 42 being reduced in size. A similar variation can be achieved for the vacuum-assisted concept according to FIG. 2, in which the surface area or the enclosed volume can be reduced or increased in size. The resilient locking can be varied in terms of retaining force by virtue of the prestressing of the spring 46 being adjusted.

FIG. 4 illustrates a further variant of the invention. The retaining device 40 is of hydraulic design and therefore forms, at the same time, a damper device 50. The retaining device 40 with the damper device 50 has a piston rod 51, which is mounted in a pivotable manner on the upper part 10. A piston 53 is arranged at the end of the piston rod 51 and is guided in a hydraulic cylinder 52, which for its part is secured in an articulated manner on the lower part 30. The piston 53 subdivides the cylinder 52 into an extension chamber and a flexion chamber. In the case of flexion, the hydraulic fluid is directed, through a bypass 54, from the flexion chamber into the extension chamber. The bypass 54 contains a nonreturn valve 55, which is prestressed via a spring. The spring prestressing can be adjusted via an adjustment device 56, for example a screw. If the flexion moment applied is one which exceeds the spring prestressing of the nonreturn valve 50, the nonreturn valve 55 opens and the hydraulic fluid can flow from the flexion chamber, through the bypass 54, into the extension chamber and, possibly, an equalizing tank. The joint, e.g. knee, elbow or ankle, bends counter to the resulting flow resistance and allows flexion. Flexion is therefore released, the flexion movement being damped. In the case of extension, the hydraulic fluid flows out of the extension chamber through a return-flow channel (not illustrated), which is likewise provided with a nonreturn valve, albeit one acting in the opposite direction, and back into the flexion chamber. The flexed position of the joint is shown in the right-hand illustration.

The flow resistance can be adjusted by the spring prestressing and the adjustment device 56; it is also possible to switch over between two levels of prestressing, and therefore also two release moments, or else to achieve different resistances in the unlocked position, which therefore allows access to flexion.

FIG. 5 illustrates a further variant of the invention, in which, for example by way of a magnetic locking mechanism, the upper part 10 can be displaced relative to the lower part 30 counter to a spring force. In the exemplary embodiment illustrated, the upper part 20 has an axis guided about the pivot pin 20 in a slot 13, which is formed in the lower part 30. The slot 13 contains a spring 60, which counteracts displacement along the slot 13. If an increased axial force is applied along the slot 13, the magnet 41 is displaced relative to the yoke 42 and reduces the retaining force between the magnet 41 and the yoke 42 and therefore also the moment of flexion which has to be applied in order to provide for flexion. Corresponding embodiments are also provided for the variants of FIGS. 2 and 3. In addition to reducing the necessary flexion moment, it is also possible for displacement of the upper part 10 in relation to the lower part 30 to increase the flexion moment which has to be applied.

FIG. 6 shows a variant of FIG. 1 in which, in addition to the retaining device 40 with a magnet 41 and a yoke 42, a hydraulic damper 50 is arranged between the upper part 10 and the lower part 30. The hydraulic damper 50 is constructed in a manner analogous to the hydraulically active retaining device 40 according to FIG. 4 and has a housing 52, in which a cylinder 53 is mounted in a displaceable manner. The cylinder 53 subdivides the housing 52 into two chambers—an extension chamber and a flexion chamber—which are coupled to one another in terms of flow via overflow channels and, possibly, equalizing tanks. It is possible to adjust the hydraulic resistance within the overflow channels; if appropriate, pressure can build up in a pressure tank in order to assist a reverse movement.

In the exemplary embodiment illustrated, the piston 53 is secured on the upper part 10 at an upper bearing joint 57 by way of a piston rod 51. The upper bearing point 57 is located posterior to the pivot pin 20. The cylinder 52 is arranged on the lower part 30 at a lower bearing point 58. If the joint is flexed from the extended position according to the left-hand illustration of FIG. 6, once the magnetically active retaining device 40 has been overcome and a sufficiently large moment of flexion has been applied about the pivot pin 20, the upper part 10 pivots relative to the lower part 30. On account of the movement of the upper bearing point 57 relative to the lower bearing point 58, the hydraulic piston 53 is moved in the direction of the lower bearing point 58. Hydraulic fluid flows from the lower chamber into the upper chamber, and the hydraulic resistance within the hydraulic damper 50 damps the flexing movement of the upper part 10 relative to the lower part 30. The hydraulic resistance and therefore also the damping of the flexing action can be adjusted, for example by manually adjustable or motor-adjustable valves or throttles within the overflow channels. In the case of a movement in the opposite direction, that is to say in the case of an extension movement, the upper bearing point 57 moves away from the lower bearing point 58, the piston 53 is moved in the opposite direction and an extension movement is correspondingly damped. It is possible to provide damping resistances of different magnitudes for the extension movement or flexion movement.

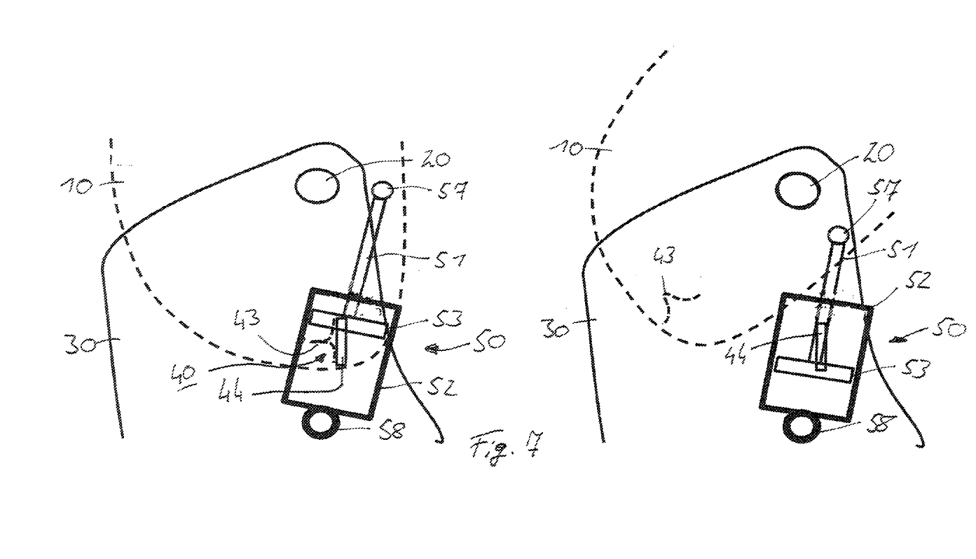

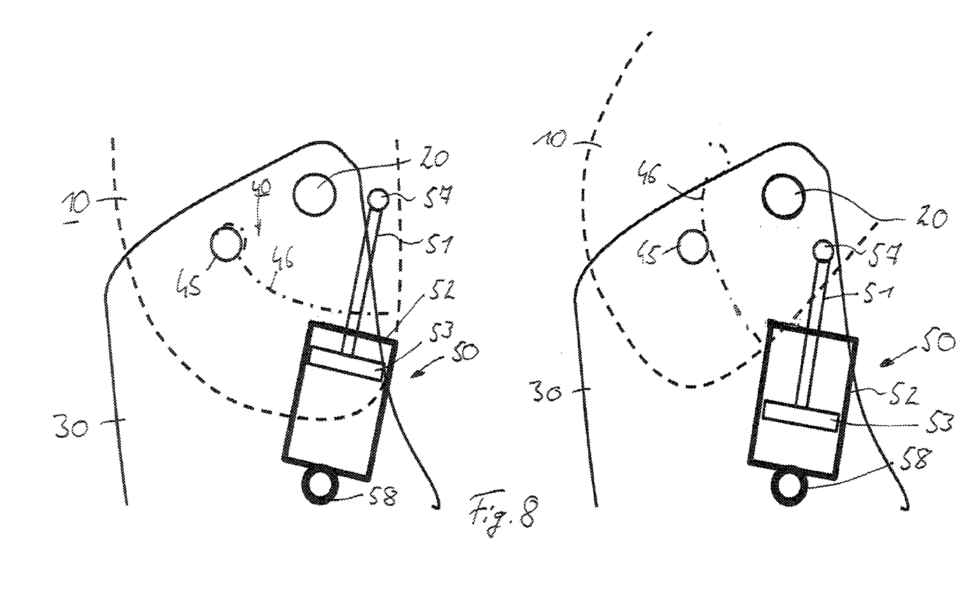

FIG. 7 shows the variant of FIG. 2 with the retaining device 40 embodied in the form of a suction cup 43 and of a smooth surface 44 in combination with the hydraulic damper 50, as has been described in FIG. 6. FIG. 8 shows a variant of FIG. 3 with the resilient retaining device 40 connected to a separate hydraulic damper 50 according to FIG. 6. What has been said in each case in relation to the retaining devices according to FIGS. 2 and 3 applies correspondingly in combination with what has been said in relation to the hydraulic damper according to FIG. 6.

A use example of the invention relates to orthoses with an abovedescribed orthopedic joint device, having a retaining device which, when an applied force is exceeded, eliminates, or vastly reduces, the resistance to flexion so that injury, e.g. in the event of spasm, is avoided. Use is made here of spring-loaded or damped orthopedic joint devices, so-called dynamic joints, in order to counteract contractions or to treat the same by way of stretching exercises. The retaining device here acts as a safeguard against overload.

1. An orthopedic joint device, comprising:
an upper part, on which are arranged upper connection members for securing the upper part to a patient;
a lower part, which is fastened on the upper part in an articulated manner about a pivot axis;
a retaining device arranged between the upper part and the lower part, wherein the retaining device has a flexion-moment-controlled design and arrests flexion movement of the lower part relative to the upper part and, when a predetermined flexion moment is exceeded, releases the lower part for flexion movement relative to the upper part;
wherein the orthopedic joint device is designed in the form of an orthotic or prosthetic knee joint, an orthotic or prosthetic elbow joint, or an orthotic or prosthetic wrist joint.

2. The orthopedic joint device as claimed in claim 1, further comprising a damper device arranged between the upper part and the lower part and damps a flexion movement and/or extension movement of the lower part relative to the upper part.

3. The orthopedic joint device as claimed in claim 2, wherein the damper device is designed such that it can be switched between a high damping resistance and a low damping resistance.

4. The orthopedic joint device as claimed in claim 3, wherein the damper device is assigned an axial-force-dependent switching device which, when an axial force acting on the lower part drops below a certain value, enables the reduced damping resistance.

5. The orthopedic joint device as claimed in claim 3, wherein the damper device is assigned a mechanical switch or a sensor-controlled switch provided with an actuator.

6. The orthopedic joint device as claimed in claim 1, wherein the retaining device is designed in the form of a force-fitting or form-fitting retaining device.

7. The orthopedic joint device as claimed in claim 1, wherein a retaining force exerted by the retaining device is adjustable.

8. The orthopedic joint device as claimed in claim 1, wherein the upper part is mounted in a displaceable manner in relation to the lower part.

9. The orthopedic joint device as claimed in claim 8, further comprising a displacement-counteracting spring element arranged between the upper part and the lower part.

10. The orthopedic joint device as claimed in claim 1, wherein the retaining device has at least one of a magnetic coupling, a suction-cup coupling, a prestressed snap spring, and a hydraulic arresting unit with a switchable arresting valve.

11. (canceled)

12. The orthopedic joint device as claimed in claim 1, further comprising a separate hydraulic damper arranged between the upper part and the lower part.

13. The orthopedic joint device as claimed in claim 1, wherein the retaining device has, or forms, a sensorless-operation release mechanism.

14. An orthopedic joint device, comprising:
an upper part having upper connection members to secure the upper part to a patient;
a lower part pivotally connected to the upper part;
a retaining device arranged between the upper part and the lower part, the retaining device having a flexion-moment-controlled design that stops flexion movement of the lower part relative to the upper part and, when a predetermined flexion moment is exceeded, releases the lower part for flexion movement relative to the upper part;
wherein the orthopedic joint device is designed in the form of an orthotic or prosthetic knee joint, an orthotic or prosthetic elbow joint, or an orthotic or prosthetic wrist joint.

15. The orthopedic joint device as claimed in claim 14, further comprising a damper device arranged between the upper part and the lower part and damps a flexion movement and/or extension movement of the lower part relative to the upper part.

16. The orthopedic joint device as claimed in claim 15, wherein the damper device is designed such that it can be switched between a high damping resistance and a low damping resistance.

17. The orthopedic joint device as claimed in claim 16, wherein the damper device is assigned an axial-force-dependent switching device which, when an axial force acting on the lower part drops below a certain value, enables the reduced damping resistance.

18. The orthopedic joint device as claimed in claim 16, wherein the damper device is assigned a mechanical switch or a sensor-controlled switch provided with an actuator.

19. The orthopedic joint device as claimed in claim 14, wherein the retaining device is designed in the form of a force-fitting or form-fitting retaining device.

20. The orthopedic joint device as claimed in claim 14, wherein a retaining force exerted by the retaining device is adjustable.

21. The orthopedic joint device as claimed in claim 14, wherein the upper part is displaceably mounted relative to the lower part.
